(19) United States
(12) Patent Application Publication
(10) Pub. No.: US 2008/0306350 A1
(43) Pub. Date: Dec. 11, 2008

(54) Methods for Implementing a Weight Loss Program

(76) Inventors: Larry David Yogel, Elkins Park, PA (US); David Katz, Ambler, PA (US)

Correspondence Address: FOX ROTHSCHILD LLP;New York, 2000 Market Street, Tenth Floor, Philadelphia, PA, 19103, US

(21) Appl. No.: 11/761,025

(22) Filed: Jun. 11, 2007

(51) Int. Cl. A61B 5/00

(52) U.S. Cl. 600/300

(57) ABSTRACT

A method for implementing a weight loss program is provided. The diet meal provider educates a health care professional about a diet meal plan or a dietary aid. The health care professional may recommend the plan to his or her patient who need to loose weight. Based on such recommendation, the patient will subscribe to receive the diet meal plan or the dietary which are supplied to the patient by the diet meal provider.

### FIELD OF THE INVENTION

This invention is related to methods for weight loss programs.

### BACKGROUND OF THE INVENTION

The number of people who are overweight or obese has been increasing sharply in the United States. Studies show that the prevalence of obesity among adults aged 20-74 years increased from 15.0% at the end of the 1970s to 32.9% in 2004. Over the same period of time, for children aged 2-5 years, the prevalence of excessive weight increased from 5.0% to 13.9%; for those aged 6-11 years, prevalence increased from 6.5% to 18.8%; and for those aged 12-19 years, prevalence increased from 5.0% to 17.4%.

It is well known that being overweight or obese increases the risk of many diseases and health conditions, such as hypertension, dyslipidemia (for example, high total cholesterol or high levels of triglycerides), type 2 diabetes, coronary heart disease, stroke, gallbladder disease, osteoarthritis, sleep apnea and respiratory problems, endometrial, breast, and colon cancers, among many others. In addition to health factors, excessive weight and obesity also have a significant economic impact. According to one study of national costs attributed to both excessive weight and obesity, medical expenses accounted for 9.1 percent of total U.S. medical expenditures in 1998 and may have reached as high as $78.5 billion. These costs continue to grow.

Some people choose to ignore the risks associated with being overweight or obese. Others understand the risks, but still do nothing to lose unwanted weight. One reason people give for failing to lose extra weight is that it is time consuming and inconvenient to prepare dietary foods. Although currently there are many companies that provide pre-cooked dietary foods, many people do not trust these companies, do not have time to search for them, or simply cannot choose the one that provides the best fit for them.

Accordingly, there is a need for a method that will make it easier for people to choose an effective diet meal plan and start losing extra weight.

### SUMMARY OF THE INVENTION

In one aspect, a method for implementing a weight loss program is provided. The method comprises providing information about a diet meal plan and a dietary aid directly to a health care professional; receiving a first request for the diet meal plan or a dietary drug wherein the request is caused by the health care professional's recommendation after evaluating the patient; and supplying the diet meal plan or the dietary aid to the patient. The method may also include a step of billing the patient or receiving the payment from the health care professional for the supplied diet meal plan.

The method may also include a step of training the health care professional's assistant about different options within the diet meal plan wherein the patient is educated by the health care professional's assistant about different options within the meal plan prior to making the request for the diet meal plan. The patient may elect to be supplied with the diet meal plan once a week. The plan may be supplied to the patient's home, doctor's office, or both.

The first request comprises the patient's selection of individual prepared breakfasts, lunches, and dinners for each day. The second request may also be submitted to alter the first request. The diet plan supplied to the patient may be modified based on the patient's weight loss progress.

This method may be used for patients suffering from one or more health conditions selected from the group of meal plans for hypertension, dyslipidemia, diabetes, coronary heart disease, stroke, gallbladder diseases, osteoarthritis, sleep apnea and respiratory problems, endometrial cancer, breast cancer, and colon cancer. The diet plan may be recommended by a health care professional which will include medical doctors, doctors of osteopathic medicine, nutritionists, dietitians, chiropractors, physical therapists, and veterinarians.

In another aspect a method for implementing a weight loss program is provided. The method comprises providing information about a diet meal plan and a dietary aid directly to an office of a health care professional; receiving information about a patient from the health care professional's office comprising the patient's information and a first request for the diet meal plan or a dietary drug wherein the request is caused by the health care professional's recommendation after evaluating the patient; in response to receiving information about the patient, creating an account for the patient; and in response to the request, causing the warehouse to fulfill the first request.

The methods may also include a step of sending a communication to the patient or the health care professional where the communication to the patient comprises communication selected from the group consisting of confirming the patient's registration, notifying the patient about an upcoming delivery, requesting additional information from the patient, billing the patients, or providing information to patient about different diet meal options, or combination thereof. In some embodiments, where the patient is billed directly by the health care professional, the communication to the health care professional may also include a request for payment for the meal plan supplied to the patient.

The first request from the patient comprises the patient's selection of individual breakfasts, lunches, and dinners for each day. The patient may also submit the second request which may alter the first request from the patient.

This method may be used for patients suffering from one or more health condition selected from the group consisting of hypertension, dyslipidemia, diabetes, coronary heart disease, stroke, gallbladder diseases, osteoarthritis, sleep apnea and respiratory problems, endometrial cancer, breast cancer, and colon cancer. The diet plan may be recommended by the health care professional including medical doctors, doctors of osteopathic medicine, nutritionists, dietitians, chiropractors, physical therapists, and veterinarians.

### BRIEF DESCRIPTION OF THE FIGURES

FIG. 1 is a flow chart of one embodiment of a method for implementing a weight loss program.

FIG. 2 illustrates in details steps 15 and 16 in the method presented in FIG. 1.

### DETAILED DESCRIPTION

A method for implementing a weight loss program is provided. In step 11, a diet plan provider provides information about a diet meal plan and a dietary aid to a health care professional.

The information provided to the health care professional may include, but is not limited to, meal selections, nutritional value of the meals, options for patients with special dietary needs, safety record of the dietary aids, and similar.

The term “health care professional” means any person legally qualified to provide health care advice or services to patients. Examples of health care professionals include, but are not limited to, medical doctors, doctors of osteopathic medicine, nutritionists, dietitians, chiropractors, physical therapists, veterinarians.

The term “diet meal plan” refers to dietary foodstuffs, whereas the term “dietary aid” refers to supplements that have a minimal caloric value but are used to enhance the effectiveness of the weight loss program. Example dietary aids include, but are not limited to, dietary drugs, fiber supplements, dietary supplements, and combinations thereof. Examples of dietary drugs include, but are not limited to, Phentermine, Diethylpropion, Phendimetrazine, Benzphetamine, Sibutramine, Rimonabant, oxyntomodulin, Fluoxetine hydrochloride, Methylphenidate, or combinations thereof. Fiber supplements may include, but are not limited to, psyllium husk, methylcellulose, inulins, vegetable gums, calcium polycarbophil, beta-glucan, wheat dextrin, or combinations thereof. Examples of dietary supplements include, but are not limited to, vitamins, minerals, herbs or other botanicals, amino acids, a concentrate, metabolite, constituent, extracts, and substances such as enzymes, organ tissues, glandulars, and metabolites.

In step 12, the patient is evaluated by a health care professional. The patient may be either a human patient or an animal. The patient may come to the doctor's office for a routine check-up, because the patient is suffering from an illness, or simply to improve his or her general condition.

In step 13, since it is well known that excessive weight and obesity may cause a number of health conditions, the health care professional may recommend that the patient lose weight as part of the treatment or prevention of health conditions. Examples of such health conditions include, but are not limited to, hypertension, dyslipidemia (for example, high total cholesterol or high levels of triglycerides), diabetes, coronary heart disease, stroke, gallbladder diseases, osteoarthritis, sleep apnea and respiratory problems, some cancers (endometrial, breast, and colon), among others. Even if the patient is not overweight or obese, the health care professional may still recommend a diet meal plan to the patient who is seeking to improve their physical condition such as an athlete trying to lose weight for a competition.

In step 13, the health care professional also tells the patient about the diet meal plan and the dietary aid obtained in step 11 to the patient. The term “patient” in this context includes the patient himself or herself, or a patient's agent such as a child's parent or an animal owner. The health care professional may also recommend that the patient see the health care professional's assistants to learn more about the diet meal plan, the dietary aid, and about subscribing to the plan. The health care professional's assistant is trained by the diet plan provider to assist the patients in selecting the plan and in subscribing to the plan.

Although the patient may order the meal plan using the World Wide Web, preferably the patient will work with the health care professional's assistants to select the appropriate diet plan. In step 14, the patient may get assistance in selecting the diet meal plan. In some embodiments, the patient may choose a plan with meals pre-selected by the diet plan provider. In other embodiments, the patient may be able to customize the meal plan by selecting specific meals that he or she prefers to eat each day. The patient may also be able to select different combinations of breakfasts, lunches and dinners for each day. For example, if a patient wants to have a dessert on Friday night for dinner, he or she may have fruit for breakfast to off-set the number of calories to be consumed at dinner.

In some embodiments, the patient may be limited to a specific diet due to the patient's pre-existing condition. Thus, it is contemplated that special diets may be provided, for example, for patients with hypertension, patients with heart diseases, patients with diabetes, children, among others. Although restricted to a specific diet due to their conditions, these patients would still be able to select individual meals within that diet. Examples of special diets include, but are not limited to, juvenile obesity diets, diabetic-low sugar diets, hypertensive-low sodium diets, high cholesterol-low fat diets, or gluten free diets.

In addition to selecting specific meals or specific diets, the patient may get assistance in selecting the menu based on the individual's weight loss goals. For example, a person who absolutely needs to lose 30 pounds before a surgery in 2 months may be put on a stricter diet than a person who needs to lose 20 pounds to minimize long term risks of being overweight. In another embodiment, a patient may not want to take the dietary aid and may prefer to receive a discount on the plan. Alternatively, a patient may only request the dietary aid.

In some embodiments, once the patient finalizes his or her menu selection, the menu selection may be submitted to the health care professional for approval. This may be especially preferable in embodiments where the patient suffers from the pre-existing condition.

In step 15, upon selection of the appropriate plan in step 14, the patient subscribes and pays for the diet plan. In preferred embodiments, the patient may pay for a 6 week or 8 week program. A discount may be offered to patients who subscribe for longer terms. For example, the patient's goal may be to lose about 40 lbs at a rate of 2 lbs per week. If the patient subscribes for the 20 weeks that he or she needs to be on the plan to achieve his or her goal, the patient may receive a discount. When subscribing to the plan, the patient may indicate the preferred method, time, and location of delivery, in addition to other preferences such as, for example, the amount of food delivered at one time. When the patient subscribes for a long term, he or she may have an option to prepay for the whole period or be invoiced at regular intervals. In step 16, the diet plan is delivered to the patient.

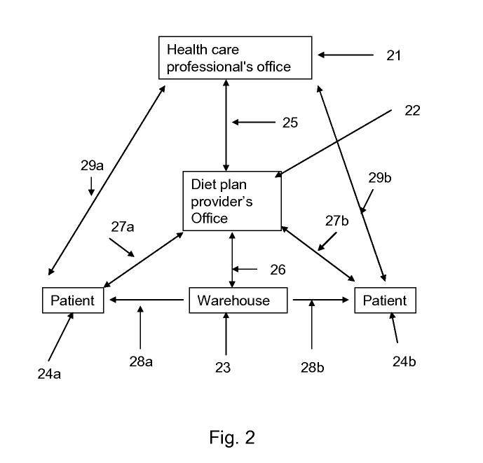

FIG. 2 describes steps 15 and 16 in more detail. Once the patient decides what plan to order, his or her information is taken in the health care professional's office 21 and is transmitted to the diet plan provider's office 22, in step 25. The information may be collected and transmitted using any known method. In one embodiment, the patient may pay for the plan using a credit card. When the credit card is swiped, the patient's payment information such as, for example, the name of the patient, his or her address, credit card number and other relevant information is automatically transmitted to the diet meal provider's office. Alternatively, the patient or the health care professionals may send this information via fax, internet or mail to the diet plan provider's office. In some embodiments, the patient may be billed directly by the health care professional's office. In these embodiments, the patient's payment information may comprise information necessary for the diet plan provider to receive payment from the health care professional's office for the supplies sent to the patient.

In addition to personal and payment information, such information as, for example, patient's delivery preferences, duration of the plan, food selections, special needs of the patient, and any other information that may be necessary to meet the patient's needs, is also submitted to the diet plan provider's office 22. Once all the information is received, an account for the patient is created by the diet plan provider.

Once the account is created, the diet plan provider sends a request to a warehouse 23, in step 26, to prepare and send packages to individual patients 24a and 24b, in steps 28a and 28b. The term “warehouse” means a facility where the food and drugs may be stored and from where the food and drugs may be dispersed. This term includes facilities used solely for storage as well as facilities that may be used for other purposes besides storage such as, for example, supermarkets that may both sell and store food and drugs. In some embodiments, this term may also include more than one facility. For example, since the facility dispensing dietary drugs must be licensed, the diet meal plan and the dietary drugs may be sent from different locations.

In steps 27a and 27b, the diet plan provider's office 22 may also send communication to the patients 24a and 24b directly over an email or telephone, for example, to confirm the patient's registration, to notify them about upcoming deliveries, request any additional relevant or updated information, update the patient about different or newly developed meal options or similar. In some embodiments, the diet plan provider may send to the patient educational materials related to the weight loss plan such as, for example, healthy cooking tips, exercise programs, general health advice, and similar.

In steps 27a and 27b, the patients 24a and 24b may also communicate directly with the diet plan provider 22 to provide or change delivery instructions or submit a request to make changes to the information specified when the patient subscribes to the diet plan. In the additional request, the patient may, for example, order additional supplies, change delivery preferences, select different foods, add or remove specific foods, change the meal plans duration, or similar.

A person with ordinary skill in the art will undoubtedly understand that at least some of the steps described above may be performed by the health care professional's office in addition to or instead of the diet meal provider. For example, in some embodiments, an account for the patient may be created and maintained by health care professional's office. In such embodiments, the patient may submit additional requests to the health care professional's office. In other embodiments, a patient may pay for the diet meal plan directly to the health care professional who, in turn, may submit the payment to the diet plan provider. It is also contemplated that in some embodiment the diet plan provider may communicate with health care professional's office in steps 29a and 29b, in addition to or instead of communicating with the patients in steps 27a and 27b.

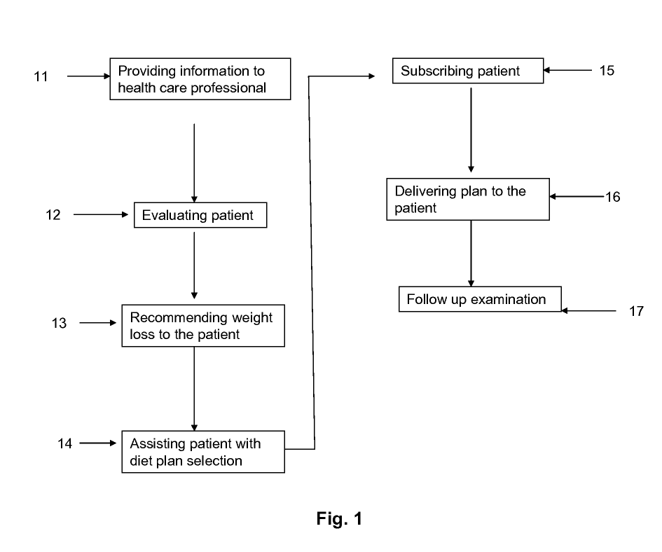

Referring back to FIG. 1, in step 16, the diet plan is delivered to the patient. In the preferred embodiment, the delivery will include a weekly supply of diet meal plans and a dietary aid. However, as mentioned before, the amount of food delivered to the patient at once may be customized to fit the needs of individual patients.

In step 17, the patient comes for a follow up examination to determine patient's progress in losing weight. In some embodiments, the patient may meet with the health care professional's assistant who can evaluate the patient's process by measuring the patient's weight, blood pressure, body mass index, or similar indicators of progress in losing weight. In other embodiments, the patient may be examined by the health care professional to ensure that, for example, there are no adverse effects on the patient's health. Based on the results of the evaluation, the patient's diet plan may be adjusted in accordance with the patient's progress. For example, if the patient is not losing enough weight, the health care professional may recommend a lower calorie intake. On the other hand, if the patient is losing weight faster than expected due to the effect of the exercise program, for example, the patient may be allowed to consume more calories. The patient's subscription is then adjusted to reflect the health care professional's recommendation.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for implementing a weight loss program, the method comprising:
providing information about a diet meal plan and a dietary aid directly to a health care professional;
receiving a first request for the diet meal plan or a dietary aid for a patient wherein the request is caused by the health care professional's recommendation after evaluating the patient;
supplying the diet meal plan or the dietary aid to the patient; and
billing and receiving a payment from the health care professional for the supplied diet meal plan.

2. The method of claim 1 wherein the first request is received from either the patient or the health care professional.

3. The method of claim 1 wherein the dietary aid is selected from the group consisting of dietary drugs, fiber supplements, dietary supplements, and combinations thereof.

4. The method of claim 1 further comprising billing the patient for the supplied diet meal plan.

5. (canceled)

6. The method of claim 1, wherein the patient is supplied with the diet meal plan once a week.

7. The method of claim 1 wherein the first request from the patient comprises the patient's selection for individual breakfast, lunch, and dinner for each day.

8. The method of claim 1 further comprising receiving a second altering the first request.

9. The method of claim 1 wherein the diet meal plan is supplied to the patient's home, doctor's office, or both.

10. The method of claim 1 further comprising modifying the diet meal plan supplied to the patient based on the patient's progress.

11. The method of claim 1, wherein the patient suffers one or more health condition selected from the group consisting of hypertension, dyslipidemia, diabetes, coronary heart disease, stroke, gallbladder diseases, osteoarthritis, sleep apnea and respiratory problems, endometrial cancer, breast cancer, and colon cancer.

12. The method of claim 1, wherein the health care professional is selected from the group consisting of medical doctors, doctors of osteopathic medicine, nutritionists, dietitians, chiropractors, physical therapists, and veterinarians.

13. The method of claim 1 further comprising training the health care professional's assistant about different options within the diet meal plan wherein the patient is educated by the health care professional's assistant about different options within the meal plan prior to making the request for the diet meal plan.

14. A method for implementing a weight loss program, the method comprising:
providing information about a diet meal plan and a dietary aid directly to a health care professional;
receiving information about a patient from the health care professional's office comprising the patient's payment information and a first request for the diet meal plan or a dietary aid wherein the request is caused by the health care professional's recommendation after evaluating the patient;
in response to receiving information about the patient, creating an account for the patient;
in response to the request from the patient, causing a warehouse to fulfill the first request by supplying the diet meal plan to the patient; and
billing and receiving a payment from the health care professional for the supplied diet meal plan.

15. The method of claim 14 farther comprising sending a communication to the patient or the health care professional wherein the communication to the patient comprises communication selected from the group consisting of confirming the patient's registration, notifying the patient about an upcoming delivery, requesting additional information from the patient, billing for the diet meal plan, and providing information to patient about different diet meal options, or combination thereof.

16. The method of claim 14 farther comprising billing the patient for the supplied diet meal plan.

17. (canceled)

18. The method of claim 14 wherein the first request comprises the patient's selection of individual breakfast, lunch, and dinner for each day.

19. The method of claim 14 farther comprising receiving a second request altering the first request.

20. The method of claim 14, wherein health care professional is selected from the group consisting of medical doctors, doctors of osteopathic medicine, nutritionists, dietitians, chiropractors, physical therapists, and veterinarians.
